(19) United States
(12) Patent Application Publication
(10) Pub. No.: US 2003/0013147 A1
(43) Pub. Date: Jan. 16, 2003

(54) Test system based on microcapillaries

(76) Inventor: Karlheinz Hildenbrand, Krefeld (DE)

Correspondence Address: BAYER CORPORATION, PATENT DEPARTMENT, 100 BAYER ROAD, PITTSBURGH, PA, 15205, US

(21) Appl. No.: 10/203,369

(22) PCT Filed: Feb. 13, 2001
(86) PCT No.: PCT/EP01/01566

(30) Foreign Application Priority Data
Feb. 25, 2000 (DE) 10008906.2

(51) Int. Cl. C12Q 1/54; C12M 1/34

(52) U.S. Cl. 435/14; 435/287.1

(57) ABSTRACT

The present invention describes a microcapillary-based test system for the determination of a component in a fluid sample, preferably an analyte in a body fluid and in particular glucose in blood.

The present invention relates to a microcapillary-based test system for determining a component in a fluid sample, preferably an analyte in a body fluid and in particular glucose and blood.

A characteristic feature of the test system described is that the sample fluid, for example whole blood or interstitial tissue fluid, is aspirated by means of capillary forces into a capillary as used, for example, in chromatography.

Self-diagnosis in the home user sector, in particular of blood glucose, has been the established state of the art for many years.

Nevertheless, continuous further development of the existing products is desirable, with the aim of achieving further improvements in accuracy and reproducibility and in particular in handling and user friendliness. Biosensors based on electrochemical detection reactions and membrane-based test strips where colour reactions are evaluated reflectometrically are state of the art.

With regard to user friendliness, in particular biosensors which aspirate the patient's blood by so-called “sip in” mechanisms are judged to be advantageous.

In order to obtain reproducible results, the total electrode compartment must be covered with blood in the case of the electrochemical sensors (cf. for example U.S. Pat. No. 5,759,364), for which purpose, as a rule, at least 3 μl of blood or more are required in the case of the products known to date.

In the case of membrane-based test strips (e.g. U.S. Pat. No. 5,453,360) which are evaluated reflectometrically, as a rule even larger amounts of blood are required.

With regard to reproducibility and precision, the uniform application of the biochemical reagent system in the test element is particularly decisive in addition to the constancy of the sensor geometry and membrane morphology.

Here, the electrochemical biosensors involve the application of an enzyme/mediator formulation, for example by screen printing or micropipetting, to a sensor electrode compartment.

In the case of the colorimetric test strip systems, microporous membranes are impregnated or coated with enzyme/indicator formulations.

Microcapillaries in numerous variations are already known from chromatography, in particular gas chromatography (GC), cf. for example “Making and Manipulating Capillary Columns for Gas Chromatography” by Kurt Grob, Heuthing Verlag, 1986.

It has now surprisingly been found that substantial progress can be made with regard to the above-mentioned target criteria on the basis of microcapillary columns which are used, for example, in chromatography.

The invention therefore relates to the use of microcapillaries in test systems for sampling by capillary forces.

According to a further aspect, the invention also relates to test systems for fluid samples, in which the sampling of the analyte is effected by means of a microcapillary. Preferably, sampling and detection of the analyte take place in the microcapillary.

The test systems according to the invention have the advantage that the sample fluid cannot come into contact with the binders or adhesives of the test format.

Suitable microcapillaries are known in particular from gas chromatography. Such gas chromatography (GC) microcapillaries fulfil a high standard of reproducibility and precision, with regard to both capillary geometry and coating of the inside of the capillary with reagents, for example polyethylene glycols for hydrophilic stationary phases or polysiloxanes for hydrophobic stationary phases. Suitable microcapillaries are also known from capillary electrophoresis.

The dimensions and material properties of the microcapillaries used according to the invention are such that they aspirate a substantially aqueous sample fluid by capillary forces. The sample volume aspirated is preferably less than 3 μl, particularly preferably less than 1 μl, very particularly preferably 0.5 μl or less.

The microcapillaries used according to the invention preferably have a round cross section. The internal diameter of the microcapillaries is preferably less than 500 μm, particularly preferably less than 250 μm, very particularly preferably 25 μm to 200 μm.

The length of the microcapillary used is preferably up to 2 cm, particularly preferably up to 1 cm, very particularly preferably about 0.5 cm.

In the case of the use, according to the invention, of microcapillaries, it is important that the microcapillary contained in the respective test format aspirates an exactly defined sample volume, depending on the measuring principle used in each case; this applies in particular to determinations of the end point of reactions, for example of enzymatic colour reactions.

GC or capillary electrophoresis columns whose dimensions are usually such that even very small amounts of sample fluids, for example 0.5 μl of blood or interstitial tissue fluid, are sufficient for a function test in the case of capillary lengths of 1 cm or less have proved suitable for the purposes according to the invention.

Owing to the small sample volumes, the capillary columns according to the invention also meet the requirements of so-called “minimally invasive” test kits, which are said to be especially advantageous, in particular to involve little pain, for the patient.

The microcapillaries consist of a suitable material which is inert under the respective conditions. Examples of these are quartz glass, standard glass and metal, such as, for example, steel.

The microcapillaries carry an internal coating, which is also referred to as the stationary phase in chromatography. Numerous materials which are known in principle from the GC columns are suitable for this coating. Hydrophilic materials, such as, for example, polyethylene glycol having various molecular weights (Carbowax®) and polyethylene glycol derivatives, for example Carbowax 4000 monostearate, are suitable. Further hydrophilic stationary phases can be prepared from polyethyleneimine, polypropylene glycol or cyclodextrins.

Hydrophobic materials, too, are suitable for the internal coating in relation to lipophilic stationary phases, siloxane polymers and siloxane copolymers being the commonest ones. Examples are dimethylpolysiloxanes, (50% cyanopropyl)-methylpolysiloxanes, (50% trifluoropropyl)-methylpolysiloxanes or 5% phenylpolycarboranepolysiloxanes.

Regarding application of the stationary phases (internal coating of the microcapillary columns), reference may be made to standard procedures known from GC columns. As described in the abovementioned literature (K. Grob, 1986), there are in principle two methods. These are static coating and dynamic coating. Capillaries of 60 to 100 m length can be coated by these procedures. The polymers of the stationary phases can also be covalently bonded to the surfaces of the quartz column, as described in the abovementioned literature.

In addition to the conventional GC columns, so-called PLOT (Porous Layer Open Tubular) columns can also be used as microcapillaries in the context of this invention. In PLOT columns, for example, alumina, silica gel, molecular sieve or porous polymers are used as stationary phases.

Further microcapillary column types which are suitable for the test elements according to the invention are capillary electrophoresis columns, which are also available with very small internal diameters of, for example, 25 μm.

The conventional capillary columns usually consist of the above-mentioned quartz (fused silica). The previously used glass columns have become much less important in chromatography but, owing to their outstanding transparency, are certainly of importance for the test elements according to the invention.

The quartz columns are provided, as a rule, on their outside, with a yellow/brown polyimide layer stable to high temperatures. Consequently, the brittleness of the quartz capillary is eliminated, and easily handled flexible systems result.

Good transparency is required for the test elements according to the invention, particularly in the case of calorimetric evaluation. Instead of the coloured polyimide coatings, it is therefore also possible to use transparent, colourless polymers, such as, for example, polysiloxanes, acrylic polymers, polyvinyl acetate, polycarbonate, polyamide or polyetherpolysulphone. If necessary, the troublesome coating can also be removed in a corresponding region of the microcapillary for optical evaluation.

An essential prerequisite for the microcapillaries as an important component of the test kits according to the invention is their property of aspirating whole blood or other test fluids with the aid of capillary forces.

It has surprisingly been found that columns having hydrophilic coatings, for example with polyethylene glycol (Carbowax®) or alumina, aspirate blood outstandingly, whereas columns modified with hydrophobic coatings (for example polysiloxane-modified) do not have this property.

The latter column types can however be modified for this purpose by aftertreatment with specific surfactants.

Suitable surfactants are ionic surfactants, for example SDS, zwitterionic surfactants, for example phospholipids, and nonionic surfactants, for example Pluronic® or fluorine surfactants (for example Bayowet FT 219®).

The sample fluid is usually a substantially aqueous fluid, in particular a body fluid. Examples are urine, interstitial tissue fluid and blood.

Typical analytes which can be determined are, for example, glucose, bilirubin, ketones, pH, proteins and cholesterol.

The detection reagents are preferably contained in the internal coating of the microcapillary (stationary phase). The measurement is usually effected through the microcapillary wall, for example colorimetrically. With regard to the integration of the analyte-specific detection reagents, the systems established in diagnostics can be used, such as, for example, detection by enzymatic or nonenzymatic colour reactions or by an electrochemical method, colour reactions, in particular enzymatic colour reactions, being preferred. Thus, various enzymatically controlled colour reactions are available, for example for the quantitative detection of blood glucose, oxygen-independent enzyme reactions being preferred for the capillary test kits described here.

For example, the glucose dehydrogenase system or the hexokinase system with tetrazolium indicators are suitable for the glucose detection.

The incorporation of the corresponding reagents can be effected either via the formulations for the stationary phases, by subsequent application as a coating on the stationary phases or by a combination of these variants.

Either the test reaction can take place heterogeneously, i.e. in the stationary phase, or the detection reagents incorporated in the stationary phase can dissolve in the sample fluid, in which case a homogeneous reaction takes place in the fluid phase and can be monitored colorimetrically.

The colour reaction can be evaluated by means of reflection, particularly with the use of PLOT columns, for example with alumina as stationary phase, or in transmission in the case of a transparent capillary system, for example GC columns with polywax as the stationary phase, it being possible to determine either the reaction kinetics or the end point of the reaction.

Depending on the type of indicator, the transmittance measurement of the colour reaction can also be effected in the whole blood sample, without separating off the red blood corpuscles beforehand.

With regard to practical handling, the microcapillary systems according to the invention should be integrated in an appropriate format. Suitable test formats are familiar in principle to a person skilled in the art. Those formats which permit optical evaluation of an enzymatic colour reaction taking place in the microcapillary are preferred.

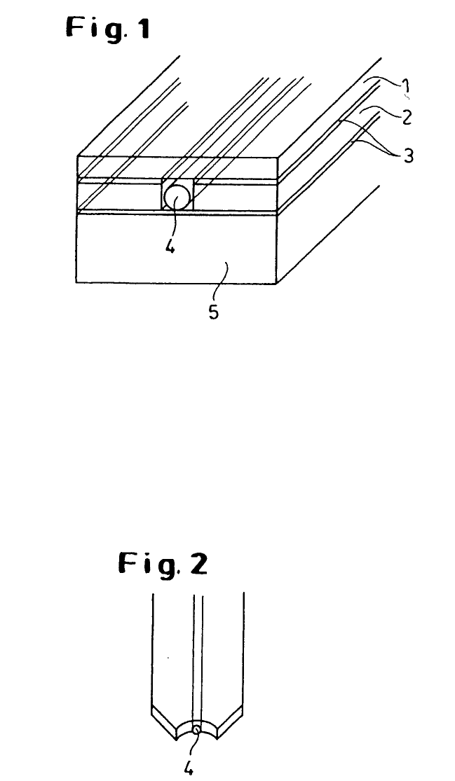

As shown in FIGS. 1 and 2, a “test strip format” which was also used for the following experiment was produced with the aid of polymer films and double-sided adhesive tapes.

FIG. 1 shows the cross section of a microcapillary test format comprising cover film (1), spacer film (2), double-sided adhesive tape (3), microcapillary (4) and base film (5).

FIG. 2 shows a microcapillary test format in plan view.

The test strip design can also be such that, by appropriate perforation, no films or adhesive tapes are present in the area where photometric evaluation is effected.

The upper cover film may be transparent so that the capillary filling process can be observed.

The same aim, namely the recording of complete filling of the capillary with sample fluid can also be achieved by using filler-containing (white) cover films which have a cut-out as an inspection window at the end of the capillary column.

In addition to medical diagnostics, applications in the area of high throughput screening are also possible for the microcapillary systems according to the invention.

Thus, perpendicular test arrays which are correspondingly arranged parallel to the geometry of microtitre plates and aspirate sample fluids from the individual microtitre plate compartments on immersion and initiate corresponding detection reactions are possible.

1. Use of microcapillaries in test systems for sampling by means of capillary forces.

2. Use according to claim 1, the microcapillary carrying a coating on the inner surface and containing the detection system for the analyte.

3. Use according to claim 1 or 2 for the determination of glucose in blood.

4. Use according to any of the preceding claims, the microcapillary consisting of glass or quartz.

5. Use according to any of the preceding claims, the internal diameter of the microcapillary being 500 μm or less.

6. Test system for fluid samples, in which the sampling is effected by means of a microcapillary.

7. Test system according to claim 6, in which the sampling and the detection of the analyte are effected in the microcapillary.

8. Test system according to claim 6 or 7, in which the aspirated sample volume is less than 3 μl.
